(19) United States
(12) Patent Application Publication
(10) Pub. No.: US 2022/0031590 A1
(43) Pub. Date: Feb. 3, 2022

(54) ORAL CARE COMPOSITION

(71) Applicant: SYMRISE AG, Holzminden (DE)

(72) Inventors: Manuel PESARO, Beverungen (Wehrden) (DE); Arnold MACHINEK, Holzminden (DE); William WADE, Clevedon Somerset (GB)

(21) Appl. No.: 16/325,802

(22) PCT Filed: Aug. 18, 2016
(86) PCT No.: PCT/EP2016/069617

§ 371 Date: Feb. 15, 2019

(51) Int. Cl. A61K 8/42; A61Q 11/00

(52) U.S. Cl. CPC A61K 8/42; A61K 2800/524; A61Q 11/00

(57) ABSTRACT

The present invention relates to oral care compositions for altering the composition of oral biofilms so that the proportion of microorganisms, which are detrimental to oral health, is reduced while the proportion of health-promoting microorganisms is increased. Furthermore, oral care products comprising the composition according to the invention in an amount sufficient to alter the composition of oral biofilms to promote dental health are provided.

The present invention relates to oral care compositions for altering the composition of oral biofilms so that the proportion of microorganisms, which are detrimental to oral health, is reduced while the proportion of health-promoting microorganisms is increased.

Furthermore, the present invention relates to the non-medical use of the oral compositions according to the invention for the treatment and/or prevention of halitosis, in particular for the suppression of Solobacterium moorei in an oral biofilm.

In addition, oral care products comprising the composition according to the invention in an amount sufficient to alter the composition of oral biofilms, so that the proportion of microorganisms, which are detrimental to oral health, is reduced while the proportion of health-promoting microorganisms is increased, are provided.

Inflammatory conditions of the gums are primarily induced by the formation of dental plaque. Colonizing bacteria form a biofilm on the surface of the teeth aided by the presence of food residues as well as components of saliva. If not sufficiently cleared away at an early stage, plaque films on the surface of the teeth result in deposition of dental calculus which is very hard to remove. The presence of raised numbers of bacteria at the gingival margin leads to inflammation of the gingivae, known as gingivitis. In susceptible individuals, gingivitis may progress to periodontitis, which can lead to tooth loss. In particular, lipopolysaccharides (LPS) present in Gram-negative bacteria can cause a non-specific immune response by LPS-stimulated macrophages, which release prostaglandin E2 (PEG2) and pro-inflammatory mediators such as interleukins and TNF-α in the affected tissue. The pro-inflammatory mediators induce the release of further PGE2s and matrix metalloproteinases (MMPs) from the residing fibroblasts, which destroy the extracellular matrix of the surrounding tissue. This allows bacteria to penetrate deeper into the tissue and promote the inflammatory process independent of the outer layer of the epithelium and the dental root causing the formation of a periodontal pocket. The alveolar bone supporting the tooth resorbs ahead of the advancing bacteria and, causing the tooth to become unstable and, if left untreated, lost.

In particular, Gram-negative anaerobic genera, including Prevotella, Porphyromonas and Fusobacterium, have been associated with the onset of gingivitis (Kistler, Booth, Bradshaw, Wade PLoS ONE 8: (2013) e71227. doi:10.1371/journal.pone.0071227; Moore, Holdeman, Smibert, Good, Burmeister, Palcanis et al., Infect Immun. 1982nd ed. 1982 November; 38(2):651-67).

Another problem associated with oral hygiene is bad breath or halitosis. While bad breath may have serious systemic causes, in most cases it results from the degradation of organic substrates such as food residue by the resident oral microorganisms. Primarily, films of anaerobic bacteria coating the tongue dorsum are considered responsible for the generation of the volatile sulfur compounds giving raise to bad breath.

In particular, Solobacterium moorei, has been found to play a significant role in the occurrence of oral malodour (Haraszthy, Journal of breath research, 2 (2008) 017002; Vancauwenberghe et al., Journal of breath research, vol. 7, no. 4 (2013)). The cited studies revealed, that a strong correlation exists between the prevalence of Solobacterium moorei in subjects with halitosis compared to subjects without halitosis as well as the H₂S production caused by the organism.

As a result, antibacterial agents are widely used in oral care products with the aim to suppress or prevent bacterial growth in the oral cavity and avoid the formation biofilms on the teeth and the oral mucosa.

It has been disclosed in US 2008/0008660 A1, that compounds of the formula 1

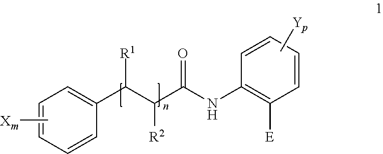

exhibit antibacterial properties and may be used to suppress the growth of a number of individual microorganisms in order to prevent bad breath. In particular, microorganisms of the genera Eubacterium, Fusobacterium, Haemophilus, Neisseria, Porphyromonas, Prevotella, Treponema and Veillonella have been postulated to be suppressed by compounds of the formula 1.

However, a number of microorganisms colonizing the oral cavity are not pathogenic and even promote oral health. Such organisms can actively protect the oral cavity against pathogenic species. Among the protective actions, general antibacterial effects against disease-associated species and the reduction or prevention of bacterial adhesion to the surface of the teeth as well as anti-inflammatory effects have been discussed in the literature.

Bacteria that have been associated with oral health include the obligate aerobes and facultative anaerobes of the genera Neisseria, Rothia, Corynebacterium and Streptococcus.

Consequently, it is highly desirable to balance the composition of the microorganisms in the oral cavity towards the health-promoting species instead of unspecifically eradicating resident bacteria.

This challenge is further complicated by the nature of complex biofilms. Biofilms consist of microorganisms growing in close association embedded in an extracellular polymeric matrix, which allows them to cooperate in various ways and provides some protection against outside influences. Bacterial species growing in a mixed-species biofilm often exhibit properties, which are not observed for the individual species grown, for example, in a liquid medium as a planktonic population. Notably, they show an enhanced resistance to antimicrobial agents such as antibiotics and disinfectants (Gilbert et al., Adv. Dent. Res., 11(1), 1997, 160-167).

A study on antimicrobial resistance in bacterial biofilms on implanted medical devices suggests that the concentration of antimicrobial agents required to reach bactericidal activity against the mixed species forming a biofilm may be at a higher order of magnitude compared to the planktonic bacteria (RodriguezMartinez and Pascual, Reviews in Medical Microbiology, 17, 2006, 65-75).

The susceptibility of microorganisms growing in biofilms to antibiotics was studied using a special technology, which allows to grow and test biofilms rapidly for effective antimicrobial agents. The Calgary Biofilm Device (CBD) provides a microtiter plate with 96 pegs on the lid, on the surface of which biofilms can be grown and which can be individually immersed into the wells of the microtiter plate. The study demonstrated that biofilms formed of pathogenic bacteria derived from several animal species are largely resistant to common veterinary antibiotics (Olson et al., The Canadian Journal of Veterinary Research, 66, 2002, 86-92).

Oral bacteria naturally form biofilms that are composed of many different species, not all of which have been cultivated (Nyvad, Fejerskov, Scand J Dent Res 95, (1987) 287-296; Dewhirst, Chen, Izard, Paster, Tanner et al., J Bacteriol. (2010) doi:10.1128/JB.00542-10). Deep sequencing studies have, for example, detected hundreds of species in dental plaque samples from individual subjects (Griffen, Beall, Campbell, Firestone, Kumar et al., Isme J 6 (2012): 1176-1185. doi:10.1038/ismej.2011.191; Abusleme, Dupuy, Dutzan, Silva, Burleson et al. Isme J 7 (2013): 1016-1025. doi:10.1038/ismej.2012.174; Kistler, Booth, Bradshaw, Wade PLoS ONE 8: (2013) e71227. doi:10.1371/journal.pone.0071227).

As a result, the antibacterial activity of any compound found against bacterial species grown in monoculture allows no reliable prognosis on how strongly or even if this compound may affect the same bacterial species in a complex biofilm, such as a natural oral biofilm.

It was therefore an object of the present invention to provide an oral care composition, which achieves a reliable antibacterial activity against microorganisms detrimental to oral health.

A further object of the present invention was to provide an oral care composition having the desired antibacterial activity against oral microorganisms detrimental to oral health while at the same time not affecting the growth of health-promoting microorganisms adversely.

Yet another object of the present invention was the provision of an oral care composition, which can be used to treat and/or prevent bad breath or halitosis.

An in-vitro oral biofilm model for oral care product evaluation has recently been developed using the commercially available Calgary Biofilm Device (CBD) (Ceri, Olson, Stremick, Read, Morck, Buret, J Clin Microbiol. (1999) June; 37(6):1771-6) seeded with a natural saliva inoculum (Kistler, Pesaro, Wade, BMC Microbiol. (2015) December; 15(1):364). The pegs of the CBD were coated in hydroxyapatite to provide a surface that was chemically similar to tooth enamel. Using a nextgeneration sequencing method, the biofilms were shown to be highly complex and similar in composition to dental plaque. In addition, the composition of the biofilms was reproducible when derived from saliva of the same individuals at different time points.

The model has been used to screen antimicrobial flavour/aroma substances for their effect on the composition of in-vitro oral biofilms. Ordination plots based on 16S rRNA gene sequence data indicated that treatment with different compounds altered the community structure of the biofilms relative to a negative control (PBS-treated biofilms). However, the differences in community structure between individual treatment groups and the negative control did not reach statistical significance by Analysis of Molecular Variance (AMOVA). It was thought that the use of only three replicates in each treatment group had resulted in insufficient statistical power to demonstrate significant differences by AMOVA. In addition, by using anaerobic incubation conditions, some health-associated aerobic/facultatively anaerobic genera found in plaque, such as Neisseria and Rothia, were absent or at very low abundance in the biofilms. Subsequent development work has shown that aerobic incubation conditions improve the growth of these aerobes or facultative anaerobes but still enable the recovery of obligate anaerobes. Aerobic conditions also more accurately represent the in-vivo oral environment

Surprisingly, it has been found out in the ensuing study that a composition comprising a compound of formula 1 and a suitable carrier selectively reduces the proportion of microorganisms detrimental to oral health while at the same time increasing the proportion of health-promoting organisms. In particular, such a selective suppression was not expected to be possible in a complex biofilm.

In contrast, treatment with thymol did not have a significant effect on the composition of in-vitro biofilms. This was surprising as thymol is a broad-spectrum antimicrobial phenolic compound and the concentration used in the study was higher than in certain mouthrinses. These findings support the fact, that such selective effects can not be expected for any known antibacterial compounds.

The above mentioned objects of the present invention are therefore met by an oral care composition comprising or consisting of:
i) a compound of the formula 1 or salt thereof or a mixture of two or more different compounds of the formula 1 and/or salts thereof

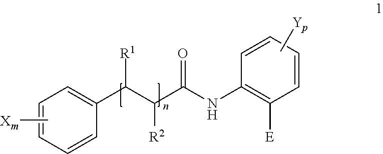

in which for the compound of formula 1 or for each compound of the formula 1 in the mixture,
m=0, 1, 2 or 3,
p=0, 1 or 2
n=0, 1 or 2,
in which if n=1 or 2 then in each case the pair R¹ and R² in each case denote H or together form a further chemical bond,
in which if m=1, 2 or 3 each X, independently of the other, denotes OH, Oalkyl or Oacyl,
in which if p=1 or 2 each Y, independently of the other, denotes OH, Oalkyl or Oacyl,
in which E=H or denotes a radical—COOR³,
R³═H or alkyl, in which R³═H also for the corresponding salts, and
ii) a suitable carrier,

for use in a method for altering the bacterial composition of oral biofilms so that the proportion of microorganisms detrimental to oral health is reduced while the proportion of health-promoting microorganisms is increased, in each case with respect to a negative control.

The oral care composition according to the present invention, is capable of altering the composition of oral biofilms as determined by sequence analysis of DNA extracted from a biofilm sample which has been treated with the composition using 16S rRNA pyrosequencing, clustering of the sequences into taxonomic units (OUTs) at a genetic distance of 0.015 and comparing the abundance of OUTs with a sample from negative control biofilms. This activity is demonstrated in example 1 below.

Unexpectedly, in the biofilms treated with the oral care composition according to the invention, the proportions of Prevotella, Veillonella, Porphyromonas, Atopobiurn, Selenomonas and Fusobacterium were reduces while the relative abundance of Neisseria, Rothia, Corynebacterium and Streptococcus were raised in comparison with untreated biofilms.

While antimicrobial activity of the compounds of formula 1 against several of the above mentioned species has been reported in the prior art, this activity has not been demonstrated to be observed in a complex biofilm. As widely recognized in the prior art, many agents known to have a strong antimicrobial activity against certain bacterial species fail to show such activity when the target species is grown as part of a multi-species biofilm. Therefore, it was highly unexpected that the compounds of formula 1 would exhibit significant antibacterial activity against certain species growing as part of a biofilm, in particular a biofilm as complex as a natural oral biofilm. Even more surprising, however, is the finding that while certain bacteria detrimental to oral health are suppressed, the growth of certain health-promoting species is at the same time not adversely affected or even encouraged. Furthermore, the carrier used in the composition according to the invention, supports the antimicrobial action by increasing the bioavailability of the compound(s) of formula 1 and facilitating their penetration into the biofilm. Advantageously, it also renders the composition more suitable to be worked into an oral care product.

The oral care composition according to the present invention is therefore able to balance the composition of the microorganisms in the oral cavity towards the health-promoting species.

According to a preferred aspect of the present invention, in the oral care composition for use as described above for one or, respectively, the, several or all compounds of formula 1: n=0, E=—COOH and m+p≤3, preferably m+p≤2, particularly preferably m+p≤1.

In a particularly preferred embodiment of the oral care composition according to the invention for use as described above, for one or, respectively, the compound of formula 1: m+p=0.

Therefore, an oral care composition for use is particularly preferred, in which one or, respectively, the compound of formula 1 is a compound of formula A:

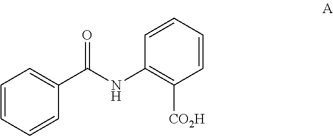 [SMILES: O=C(Nc1ccccc1C(=O)O)c1ccccc1]

The compound(s) of formula 1, wherein n=0, E=—COOH and m+p≤3, and in particular the compound of formula A, have been found to be particularly suitable to provide the desired antibacterial effects in an oral care composition according to the invention.

According to a preferred aspect of the present invention, the oral care composition as described above is intended for reducing the proportions of one or more of the bacteria selected from Prevotella, Veillonella, Porphyromonas, Atopobium, Selenomonas and Fusobacterium and increasing the proportions of one or more of the bacteria selected from Neisseria, Rothia, Corynebacterium und Streptococcus, in each case with respect to a negative control.

The oral care composition according to the present invention has been found to reduce the proportions of one or more of the bacteria selected from Prevotella, Veillonella, Porphyromonas, Atopobium, Selenomonas and Fusobacterium and increase the proportions of one or more of the bacteria selected from Neisseria, Rothia, Corynebacterium und Streptococcus, as determined by sequence analysis of DNA extracted from a biofilm sample which has been treated with the composition using 16S rRNA pyrosequencing, clustering of the sequences into taxonomic units (OUTs) at a genetic distance of 0.015 and comparing the abundance of OUTs with a sample from negative control biofilms. This activity is demonstrated in example 1 below.

Biofilms treated with the composition according to the invention had a significantly different community membership to those treated with the negative control. The OTUs that had an increased relative abundance in biofilms treated with the oral care composition according to the invention were obligate aerobes and facultative anaerobes of the genera Neisseria, Rothia, Corynebacterium and Streptococcus. These organisms have been associated with periodontal health in nextgeneration sequencing-based studies comparing the oral microbiome in health and disease (Griffen, Beall, Campbell, Firestone, Kumar, Yang et al., Isme J. 2011 ed. 2012 June; 6(6):1176-85; Kistler, Booth, Bradshaw, Wade, PLoS ONE 2013 ed. 2013; 8(8):e71227; Abusleme, Dupuy, Dutzan, Silva, Burleson, Strausbaugh et al., Isme J. 2013 ed. 2013 Jan. 10; 7(7):1016-25). Obligate anaerobes such as Prevotella spp., Porphyromonas spp., and Fusobacterium spp., were lower in relative abundance in the biofilms treated with a composition according to the invention than in the control. Increasing proportions of these taxa in plaque in the absence of oral hygiene have been associated with the onset of gingivitis (Kistler, Booth, Bradshaw, Wade, PLoS ONE, 2013 ed. 2013; 8(8):e71227; Moore, Holdeman, Smibert, Good, Burmeister, Palcanis et al. Infect Immun. 1982nd ed. 1982 November; 38(2):651-67). This confirms that the composition according to the invention promotes periodontal health.

According to a further preferred aspect of the present invention, in the oral care composition for use as described above, the carrier is selected from the group consisting of oils, alcohols, diols, polyols, phenols or esters with good solubilizing properties, preferably selected from the group consisting of ethanol, propanol, isopropanol, propylene glycol, dipropylene glycol, glycerol, ethylene glycol, 1,3-propanediol, pentylene glycol, 1,2-hexanediol, hexylene glycol, phenoxyethanol, benzyl alcohol, ethyl lactate, butyl lactate, ethylbutyrate, menthyl acetate, carvacrol, methylsalicylate, eugenol, menthone, carvone, anethole, cinnamic aldehyde, limonene, ethylacetate, isoamylacetate, diethylmalonate, peppermint oil, spearmint oil, clove oil and cinnamon oil, particularly preferably selected from propylene glycol, propanol, benzyl alcohol, diethylmalonate, butyl lactate, peppermint oil and spearmint oil.

Preferably, in an oral care composition for use (final application, e.g. toothpaste, mouthwash) according to the invention, the total amount of the compound(s) of the formula 1 and/or salt(s) thereof is in the range from 0.0005 to 1 wt. %, preferably from 0.001 to 0.5 wt. %, particularly preferably from 0.005 to 0.2 wt. %, in each case with respect to the total weight of the composition.

In order to achieve the optimal desired effect on the composition of oral biofilms, an oral care product according to the invention comprises a certain amount of the compound of formula 1. In particular, the amounts specified above have been demonstrated to be suitable to achieve the inventive effect. The amount of the compound(s) of formula 1 with respect to the amount of carrier present in the composition according to the invention depends primarily on the solubility of the compounds of formula 1 in the carrier substance. Preferably, a range of 1 to 25% of compound(s) of formula 1 with respect to the carrier is used.

Preferably, the compound of formula 1 is pre-dissolved in the carrier before it is added to the oral care composition. Typically, about 5 wt.-% of compound of formula 1 are pre-dissolved in the carrier. The final concentration of the carrier comprising the compound of formula 1 in the oral care product is then in the range of 0.1 to 1 wt.-% and the final concentration of the compound of formula 1 in the oral care product is in the range from 0.005 to 0.05 wt.-%.

In a further preferred aspect, the present invention relates to an oral care product or product for nutrition or pleasure for use in a method for altering the bacterial composition of oral biofilms so that the proportion of microorganisms detrimental to oral health is reduced while the proportion of health-promoting microorganisms is increased, in each case with respect to a negative control, comprising or consisting of an oral care composition as described above, wherein the total amount of the compound(s) of the formula 1 and/or salt(s) thereof is sufficient to reduce the proportions of one or more of the bacteria selected from Prevotella, Veillonella, Porphyromonas, Atopobium, Selenomonas and Fusobacterium and to increase the proportions of one or more of the bacteria selected from Neisseria, Rothia, Corynebacterium and Streptococcus, in each case with respect to a negative control.

The oral care composition for use as described above may advantageously be included in a variety of oral care products and confer its health-promoting effects on such products. Products with the desired activity may be found in the examples.

A composition or product for use according to the invention may further comprise one or more components selected from the group consisting of excipients and further active ingredients such as, for example, active agents from the group of non-steroidal antiphlogistics, antibiotics, steroids, anti-TNF-alpha antibodies or other biotechnologically produced active agents and/or substances as well as analgetics, dexpanthenol, prednisolon, polyvidon iodide, chlorhexidine-bis-Dgluconate, hexetidine, triclosan, benzydamine HCl, lidocaine, benzocaine, macrogol lauryl ether, benzocaine in combination with cetidyl pyridinium chloride or macrogol lauryl ether in combination with protein free hemodialysate from calf blood, as well as for example fillers (e.g. cellulose, calcium carbonate), plasticizer or flow improves (e.g. talcum, magnesium stearate), coatings (e.g. polyvinyl acetate phtalate, hydroxyl propyl methyl cellulose phtalate), disintegrants (e.g. starch, cross-linking polyvinyl pyrrolidone), softener (e.g. triethyl citrate, dibutyl phthalate) substances for granulation (lactose, gelatin), retardation (e.g. poly (meth)acrylic acid methyl/ethyl/2-trimethyl aminomethyl ester copolymerizates in dispersion, vinyl acetate/crotonic acid copolymerizates), compaction (e.g. microcrystalline cellulose, lactose), solvents, suspending or dispersing agents (e.g. water, ethanol), emulsifiers (e.g. cetyl alcohol, lecithin, sodium lauryl sulfate, PEG 40 hydrogenated castor oil), substances for modifying the rheological properties (silica, sodium alginate), substances for microbial stabilization (e.g. benzalkonium chloride, potassium sorbate, sodium benzoate, methylparaben), preservatives and antioxidants (e.g. DL-alpha-tocopherol, ascorbic acid) substances for modifying pH (lactic acid, citric acid), blowing agents or inert gases (e.g. fluorinated chlorinated hydrocarbons, carbon dioxide), dyes (iron oxide, titanium oxide), basic ingredients for ointment (e.g. paraffines, bees wax) and others as described in the literature (e.g. in Schmidt, Christin. Wirk- und Hilfsstoffe für Rezeptur, Defektur und Großherstellung. 1999; Wissenschaftliche Verlagsgesellschaft mbH Stuttgart oder Bauer, Frömming Führer. Lehrbuch der Pharmazeutischen Technologie. 8. Auflage, 2006. Wissenschaftliche Verlagsgesellschaft mbH Stuttgart).

A composition or oral care product for use according to the present invention may also be coated or encapsulated.

Encapsulation of a composition according to the invention may have the advantage of allowing a controlled release, for example upon contact with water, or a continuous release over an extended period of time. Moreover, the composition may be protected from degradation improving the shelf life of the product. Methods for encapsulation of active ingredients are well known in the art and a number of encapsulation materials as well as methods how to apply them to a composition according to specific requirements are available.

Furthermore, a composition or product for use according to the invention may be in the form of a solution, suspension, emulsion, tablets, granules, powder or capsules.

The oral care product or product for nutrition or pleasure for use according to the invention may be selected from the group consisting of tooth paste, tooth powder, tooth gel, tooth cleaning liquid, tooth cleaning foam, mouth wash, mouth rinse, mouth spray, dental floss, chewing gum and lozenges.

Such compositions or products may contain abrasive systems (abrasive and/or polishing components) such as silicates, calcium carbonate, calcium phosphate, aluminum oxide and/or hydroxyl apatite, surfactants such as e.g. sodium lauryl sulfate, sodium lauryl sarcosinate and/or cocamidopropyl betaine, humectants such as glycerol and/or sorbitol, thickening agents, e.g. carboxy methyl cellulose, poly ethylene glycols, carrageenans and/or Laponite®, sweeteners such as saccharine, aroma and taste correcting agents for unpleasant taste impressions, taste modifying substances (e.g. inositol phosphate, nucleotides, e.g. guanosine monophosphate, adenosine monophosphate or other substances, e.g. sodium glutamate or 2-phenoxy propionic acid), cooling agents such as menthol derivates (e.g. L-mentyl lactate, L-menthyl alkyl carbonate, menthone ketals), icilin and icilin derivates, stabilizers and active agents such as sodium fluoride, sodium monofluoro phosphate, tin difluoride, quarternary ammonium fluorides, zinc citrate, zinc sulfate, tin pyrophosphate, tin dichloride, mixtures of different pyrophosphates, triclosane, cetyl pyridinium chloride, aluminum lactate, potassium citrate, potassium nitrate, potassium chloride, strontium chloride, hydrogen peroxide, aroma substances, sodium bicarbonate and/or smell correcting agents.

Chewing gums or dental care chewing gums may comprise a chewing gum base comprising elastomers, e.g. polyvinyl acetate (PVA), polyethylene, (low or medium molecular) polyiso butane (PIB), polybutadiene, isobutene/isoprene copolymers, polyvinyl ethyl ether (PVE), polyvinyl butyl ether, copolymers of vinyl esters and vinyl ethers, styrene/butadiene copolymers (SBR) or vinyl elastomers, e.g. based on vinyl acetate/vinyl laurate, vinyl acetate/vinyl stearate or ethylene/vinyl acetate and mixtures of the mentioned elastomers as e.g. example described EP 0 242 325, U.S. Pat. Nos. 4,518,615, 5,093,136, 5,266,336 5,601,858 or U.S. Pat. No. 6,986,709. Additionally chewing gum bases may contain further ingredients, e.g. (mineral) filers, e.g. calcium carbonate, titanium dioxide, silicone dioxide, talcum, aluminum oxide, dicalcium phosphate, tricalcium phosphate, magnesium hydroxide and mixtures thereof, plasticisers (e.g. lanolin, stearic acid, sodium stearate, ethyl acetate, diacetin (glycerol diacetate), triacetin (glycerol triacetate) and trietyhl citrate), emulsifiers (e.g. phosphatides, such as lecithin and mono and diglycerides of fatty acids, e.g. glycerol monostearate), antioxidants, waxes (e.g. paraffine waxes, candelilla waxes, carnauba waxes, microcrystalline waxes and polyethylene waxes), fats or fatty oils (e.g. hardened (hydrogenated) plant or animal fats) and mono, di or triglycerides.

The present invention further relates to an oral care composition comprising or consisting of
i) a compound of formula A or salt thereof,

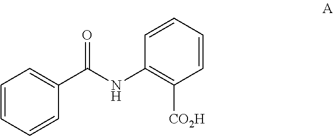 [SMILES: O=C(Nc1ccccc1C(=O)O)c1ccccc1]

and
ii) a carrier selected from oils, alcohols, diols, polyols, phenols or esters with good solubilizing properties, preferably selected from the group consisting of ethanol, propanol, isopropanol, propylene glycol, dipropylene glycol, glycerol, ethylene glycol, 1,3-propanediol, pentylene glycol, 1,2-hexanediol, hexylene glycol, phenoxyethanol, benzyl alcohol, ethyl lactate, butyl lactate, ethylbutyrate, menthyl acetate, carvacrol, methylsalicylate, eugenol, menthone, carvone, anethole, cinnamic aldehyde, limonene, ethylacetate, isoamylacetate, diethylmalonate, peppermint oil, spearmint oil, clove oil and cinnamon oil, particularly preferably selected from propylene glycol, propanol, benzyl alcohol, diethylmalonate, butyl lactate, peppermint oil and spearmint oil.

As already mentioned above, the compound of formula A has been found to be particularly effective in providing the desired antibacterial effects when combined with a suitable carrier in an oral care composition according to the invention. In particular the carriers recited as preferably above have been found to enhance the advantageous selective altering effect on the bacterial composition of oral biofilms as described above.

In the oral care composition described above, the amount of the compound of formula A with respect to the amount of carrier depends primarily on the solubility of the compound of formula A in the carrier substance. For the preferred carriers, a range of 1 to 25%, preferably 1 to 10%, of compound of formula A with respect to the carrier has been found to be advantageous.

The present invention also relates to the non-medical use of an oral care composition as defined above for the treatment and/or prevention of halitosis.

In particular, the present invention relates to the non-medical use defined above, wherein Solobacterium moorei is suppressed in an oral biofilm.

Advantageously, the oral care composition as described above has been demonstrated to specifically suppress the growth of Solobacterium moorei, which has been associated with non-pathologic halitosis or bad breath. Therefore, the composition according to the invention may be used to prevent halitosis or bad breath.

The following examples are added to illustrate the present invention without being intended to limit the scope. In particular, the compound of formula 1 used in the examples can be substituted by any other or a mixture of compound(s) of formula 1.

### SHORT DESCRIPTION OF THE FIGURES

FIG. 1 shows the OTUs that were significantly differentially abundant between biofilms treated with the composition according to the invention and those treated with a negative control using Linear Discriminant Analysis Effect Size (LEfSe). Bars with a positive LDA score (black bars) represent the OTUs that are most significantly associated with samples treated with the composition according to the invention, those with a negative LDA score (white bars) represent the OTUs that are most significantly associated with control samples.

FIGS. 2 a) and 2 b) show the relative abundance of OTUs showing greatest differences between treatments with the composition according to the invention (black columns) and control treatments (white columns).

FIG. 3 shows a heat map comparing samples based on the predominant genera detected (≥1%). The samples optD refer to compositions according to the invention.

FIG. 4 shows the relative abundance of genera in control treatment groups (left column) and treatment groups treated with the composition according to the invention (right column).

### EXAMPLE 1: TESTING THE EFFECT OF TREATMENT WITH ACTIVE AGENTS ON THE COMPOSITION of In-Vitro Oral Biofilms

Participants

Six volunteers were recruited for saliva donation. Participants were between 18 and 65 years of age and were medically healthy volunteers. Subjects with systemic conditions that may have affected their immune or inflammatory status, and/or had taken antibiotics less than one month prior to saliva collection were excluded from the study. There was no selection based on gender. Volunteers were asked to refrain from eating or drinking for one hour before donating saliva.

Inoculation of the Calgary Biofilm device (CBD)

Approximately 5 ml of saliva was collected from each volunteer by expectoration into sterile universal tubes. The saliva samples were then pooled together in equal volumes. DNA was extracted from an aliquot of the pooled saliva and 200 μl was pipetted into each well of a microtitre plate, as required. The wells around the edge of the microplate were not used. The device lid, with 96 hydroxyapatite-coated pegs (to mimic teeth) was fitted so that the pegs were bathed in saliva. The CBD was then incubated for 18 hours at 37° C. in air+5% CO₂ after which the lid was transferred to a new baseplate containing Brain Heart Infusion (BHI) broth (Fluka Analytical) supplemented with hog gastric mucin (1 g/L), haemin (10 mg/L), and vitamin K (0.5 mg/L). The growth medium was changed after every 3.5 days.

Preparation of Active Test Agents

The active agents were prepared as follows: 5% or 7.5% stock solutions were made in absolute ethanol. The stock solutions were then diluted to working concentration in sterile PBS. Thymol was diluted down to a final concentration of 0.1% v/v and the composition according to the invention, comprising 95% carrier and 5% of the compound of formula A, was diluted to 0.15% v/v. The final test concentration of the compound of formula A was 0.0075 wt.-%.

Treatment of the Biofilms

At 7 days, biofilms were treated with the active agents or a negative PBS control. Treatments were performed twice daily, at 9 am and 5 pm, for seven days. The pegs with biofilms were immersed into 200-μl aliquots of the test substances in a 96-well microplate and placed on a shaker with gentle agitation for 30 s. Pegs were then washed in PBS for a further 30 s on the shaker before returning them to the growth medium.

A total of 10 replicate samples were included in each treatment group; biofilms from three pegs were used for a single sample and six CBD plates were required in total.

Removal of Pegs and Propidium Monoazide Treatment of Samples for Pyrosequencinq Analysis

At 14 days, pegs with biofilms were snapped off the lid with sterile pliers and washed by dipping into sterile PBS three times. All of the visible biofilm material was removed using a sterile curette and suspended into 500 μl of PBS. Each sample was subjected to propidium monoazide (PMA) treatment to prevent subsequent PCR amplification of extracellular DNA and DNA from dead or damaged cells: 1.25 μl of PMA was added (at a final concentration of 50 μM) to the cells suspended in PBS and incubated in the dark with occasional shaking for 5 mins at room temperature. The samples were then exposed to light from a 500 W halogen lamp for 5 mins at a distance of 20 cm. During the exposure time the samples were placed on ice to avoid excessive heating and subjected to occasional shaking. The samples were used for DNA extractions immediately after the PMA treatment.

DNA Extraction

DNA was extracted from the pooled saliva and the biofilm samples using the GenElute Bacterial DNA extraction kit (Sigma-Aldrich). DNA extraction was performed following the manufacturer's instructions with an additional cell lysis step to increase the recovery of DNA from Gram-positive cells, in which samples were incubated in a 45 mg/ml lysozyme solution at 37° C. for 30 mins.

Pyrosequencing of 16S rRNA Genes

The bacterial composition of the biofilms and saliva was determined using 454 pyrosequencing of partial 16S rRNA genes. PCR amplification of a fragment of the 16S rRNA gene, approximately 500 bp in length covering the V1-V3 hypervariable regions, was performed for each DNA sample using composite fusion primers. The fusion primers were comprised of the broad-range 16S rRNA gene primers 27 FYM and 519 R along with Roche GS-FLX Titanium Series adapter sequences (A and B) for 454-pyrosequencing using the Lib-L emulsion-PCR method. The forward primers included previously described 12-base error-correcting Golay barcodes. PCR reactions were performed using Extensor Hi-fidelity PCR mastermix (Thermo-Scientific) along with the appropriate barcoded forward primer and the reverse primer. The PCR conditions were as follows: 5 mins initial denaturation at 95° C., followed by 25 cycles of 95° C. for 45 s, 53° C. for 45 s and 72° C. for 45 s and a final extension of 72° C. of 5 mins. PCR amplicons was then purified using the QIAquick PCR purification kit (Qiagen) according to the manufacturer's instructions. The size and purity of the amplicons was checked using the Agilent DNA 1000 kit and the Agilent 2100 Bioanalyzer. Quantitation of the amplicons was performed by means of a fluorometric assay using the Quant-iT Picogreen fluorescent nucleic acid stain (Invitrogen). The amplicons were then pooled together at equimolar concentrations (1×10⁹ molecules/μl). Emulsion-PCR and unidirectional sequencing of the samples was performed using the Lib-L kit and the Roche 454 GS-FLX+Titanium series sequencer by the Department of Biochemistry, Cambridge University, Cambridge, UK.

Sequence Analysis

Sequence analysis was performed using the ‘mothur’ software suite, following the 454 standard operating procedure on mothur.org. The sequences were denoised using the AmpliconNoise algorithm, as implemented by mothur. Sequences that were less than 440 bases in length and/or have one of the following: >2 mismatches to the primer, >1 mismatch to the barcode regions, and homopolymers of >8 bases in length, were discarded. The remaining sequences were trimmed to remove primers and barcodes and aligned to the SILVA 16S rRNA reference alignment. The UChime algorithm was used to identify chimeric sequences, which were removed from the dataset. Sequences were clustered into operational taxonomic units (OTUs) at a genetic distance of 0.015 (approximately species level) using the average neighbour algorithm and identified using a Naïve Bayesian classifier with the Human Oral Microbiome Database (HOMD) reference set (version 13).

Statistical Analysis

The bacterial community composition of thymol treated biofilms and biofilms treated with the composition according to the invention was compared to that of the negative control biofilms using principal coordinates analysis (PCoA) plots based on thetaYC and Jaccard index distance matrices. AMOVA was used to determine if there were statistically significant differences between the communities exposed to the antimicrobials and the negative control. Linear Discriminant Analysis Effect Size (LEfSe) was used to detect OTUs that were significantly differentially abundant between the different treatment groups and the negative control. A heatmap comparing biofilms based on the relative abundances of the predominant genera was generated in ‘R’ using the ‘vegan’ package.

Results

Pyrosequencing yielded 467,854 sequences after quality filtering and chimera removal. The biofilm samples were sub-sampled to 7101 sequences for OTU-based comparisons. A mean of 243 (±55) species-level OTUs was detected in the biofilm samples whilst 533 OTUs were detected in the pooled saliva sample. There were no significant differences in the richness or diversity of the biofilm samples in the different treatment groups (Kruskal Wallis test, Table 1). The predominant OTUs detected in the biofilms were Streptococcus anginosus, Prevotella oralis and Veillonella parvula.

TABLE 1
Richness and diversity of microbial communities
in control and treated biofilm samples

| | Mean no. of OTUs | Inverse Shannon index |
|---|---|---|
| | (sd) | (sd) |
| Negative control | 261.7 | 5.7 |
| | 58.9 | 2.3 |
| Thymol | 250.8 | 5.5 |
| | 45.9 | 2.3 |
| Composition according | 224.5 | 6.3 |
| to the invention | 45.3 | 2.8 |

PCoA plots comparing the biofilms based on community membership (Jaccard index) and structure (thetaYC) were analyzed. Biofilms treated with thymol did not cluster separately from those treated with the negative control. The composition according to the invention, however, did indicate an effect with most of the biofilm replicates clustering separately to the negative control replicates. AMOVA showed no overall statistically significant difference in community structure among treatment groups and therefore did not progress to pairwise comparisons. There was, however, an overall significant difference in community membership among treatment groups by AMOVA (P=0.001). Biofilms treated with the composition according to the invention were found to be significantly different to the negative control in the pairwise comparisons (P=0.006), whilst thymol—even though it was used at a higher concentration—showed no significant differences.

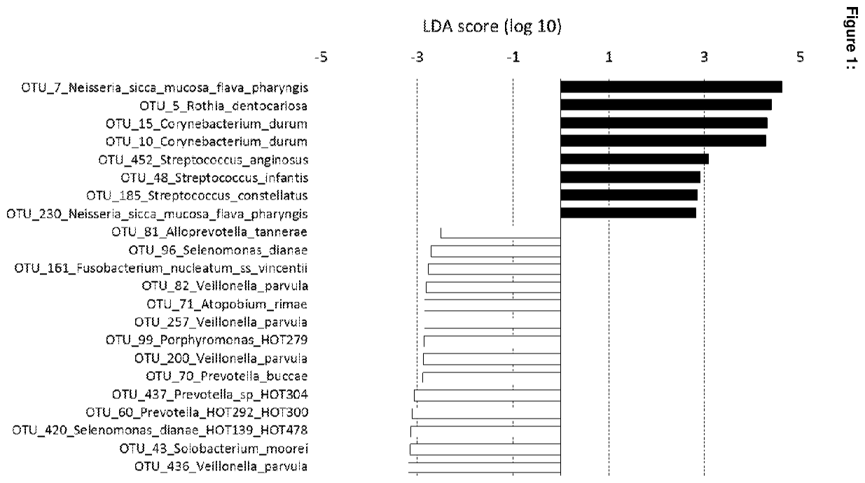

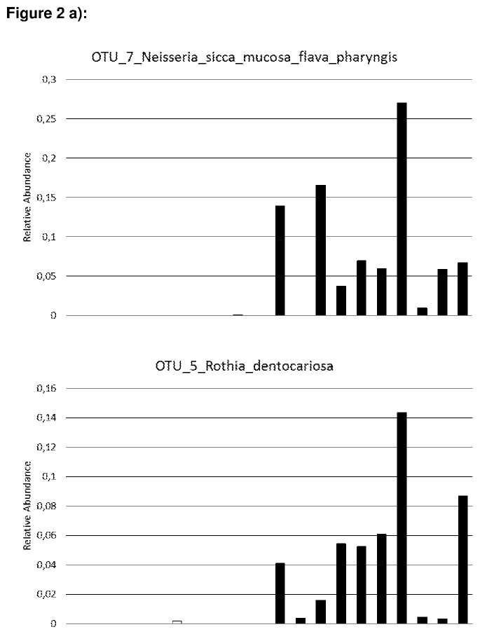

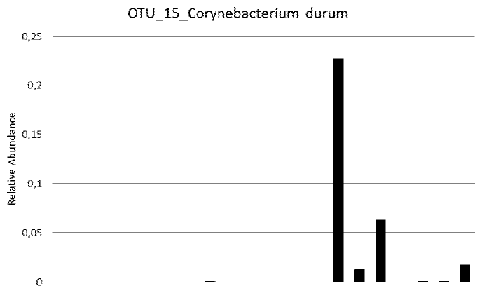

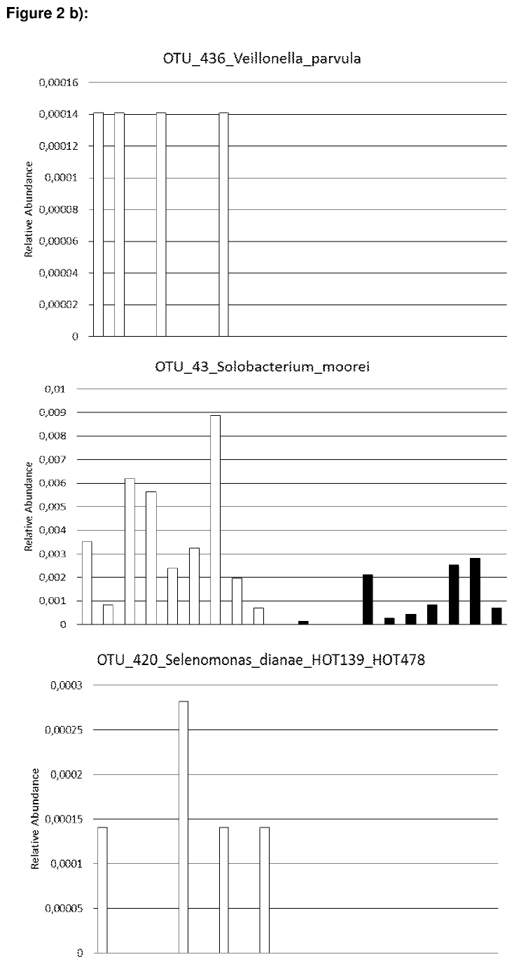

LEfSe detected 22 significantly differentially abundant OTUs between the biofilms treated with the composition according to the invention and negative control biofilms (FIG. 1). A Veillonella parvula OTU was most significantly associated with the negative control, whilst a Neisseria sicca/mucosa/flava/pharyngis OTU was most strongly associated with biofilms treated with the composition according to the invention (FIGS. 2a and 2b).

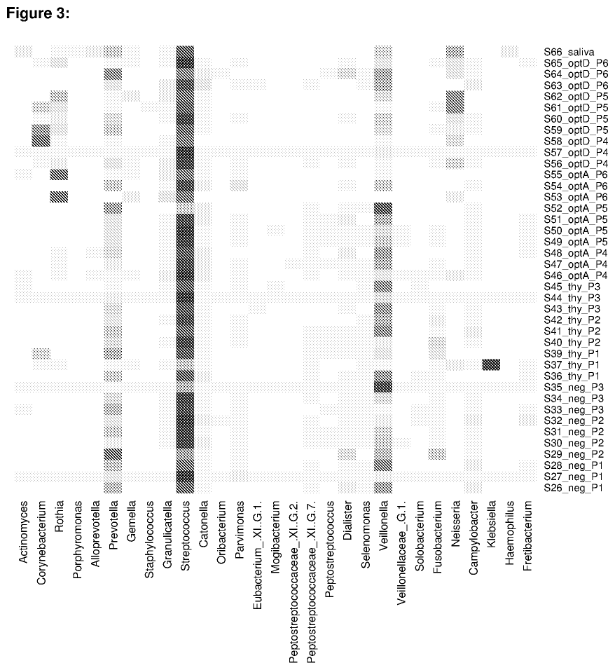

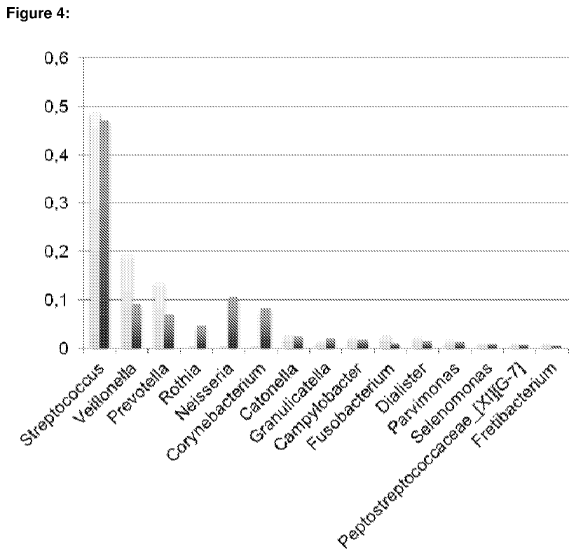

The predominant genera (≥1% relative abundance) detected in the biofilms and the pooled saliva inoculum are shown in a heatmap (FIG. 3). The predominant genera in the majority of biofilms and those treated with the negative control were Streptococcus, Veillonella and Prevotella. In a number of the biofilms treated with antimicrobials, Corynebacterium, Rothia and Neisseria were among the dominant genera. However, there was high variability in the abundances of these genera among replicates in the same treatment group. FIG. 4 shows the relative abundance of genera in the biofilms treated with the composition according to the invention and control biofilms alone and confirms the finding that the proportions of anaerobic genera such as Prevotella and Veillonella were reduced by the treatment with the composition according to the invention, whilst the relative abundance of aerobic genera such as Neisseria, Rothia and Corynebacterium were raised as a result.

### EXAMPLE 2: PEPPERMINT FLAVOUR PF1 (AMOUNTS IN % O B.W.)

| Ingredients | Amount |
|---|---|
| Isobutyraldehyde | 0.5 |
| 3-Octanol | 0.5 |
| Dimethyl sulphide | 0.5 |
| trans-2-Hexenal | 1.0 |
| cis-3-Hexenol | 1.0 |
| 4-Terpineol. natural | 1.0 |
| Isopulegol | 1.0 |
| Piperitone. natural. from eucalyptus | 2.0 |
| Linalool | 3.0 |
| 8-Ocimenyl acetate. 10% in triacetin | 5.0 |
| Isoamyl alcohol | 10.0 |
| Isovaleraldehyde | 10.0 |
| alpha-Pinene. natural | 25.0 |
| beta-Pinene. natural | 25.0 |
| Neomenthol. racemic | 40.0 |
| Eucalyptol (1,8-cineol). natural | 50.0 |
| L-Menthyl acetate of the formula D | 70.0 |
| L-Menthone | 220.0 |
| D-Isomenthone | 50.0 |
| L-Menthol | 483.5 |
| Nonenolide | 1.0 |

### EXAMPLE 3: WINTERGREEN FLAVOR PF2 (AMOUNTS IN % B.W.)

| Ingredients | Amount |
|---|---|
| dipropylene glycol | 8 |
| Anethole | 9 |
| I-menthol (natural or synthetic) | 45 |
| Peppermint oil piperita type | 2 |
| Peppermint oil arvensis type | 3 |
| Spearmint oil spicata type | 1 |
| Eugenol | 7 |
| Eucalyptol | 5 |
| Methyl salicylate | 20 |

### EXAMPLE 4: ISOAMYLACETATE TYPE FLAVOR PF3 (AMOUNTS IN % B.W.)

| Ingredients | Amount I | Amount II |
|---|---|---|
| Isoamylacetate | 2 | 2 |
| Ethylbutyrate | 0.5 | — |
| Butylbutyrate | — | 0.5 |
| Ethyl vanillin | 2 | — |
| Vanillin | — | 1 |
| Frambinon TM [4-(4-hydroxyphenyl)-2-butanon] | 0.5 | 0.5 |
| I-menthol | 8 | 11 |
| Triacetin | | 85 |
| 1.2-propylene glycol | 87 | |

### EXAMPLE 5: CINNAMON TYPE COOL FLAVOR PF4 (AMOUNTS IN % B.W.)

| Ingredients | Amount |
|---|---|
| dipropylene glycol | 3 |
| Menthlymethylether | 3 |
| Cinnamaldehyde | 10 |
| Anethole | 9 |
| Eugenol | 2 |
| I-menthol | 40 |
| Peppermint oil piperita type | 10 |
| Peppermint oil arvensis type | 10 |
| Spearmint oil spicata type | 8 |
| (1R.2S.5R)-N-ethyl-2-isopropyl-5-methylcyclohexane- | 2 |
| carboxamide (WS-3) | |
| (1R.2S.5R)-N-[4-cyanomethylphenyl]-2-isopropyl-5- | 0.5 |
| methylcyclohexane-carboxamide | |
| Menthone glycerol ketal (Frescolat MGA ®) | 1.5 |
| Menthol propylene glycol carbonate (Frescolat MPC ®) | 1.5 |

### EXAMPLE 6:TOOTHPASTE (AMOUNTS IN % B.W.)

| Ingredients | Amount |
|---|---|
| Water (deionized) | Ad 100 |
| Sorbitol 70% | 45.00 |
| Trisodiumphosphate | 0.10 |
| Saccharin | 0.20 |
| Sodiummonofluorophosphate | 1.14 |
| PEG 1500 | 5.00 |
| Sident 9 (abrasive silica) | 10.00 |
| Sident 22 S (Thickening silica) | 8.00 |
| Sodiumcarboxymethylcellulose | 1.10 |
| Titanium (IV) oxide | 0.50 |
| Water (deionized) | 4.50 |
| Sodiumlaurylsulfate (SLS) | 1.50 |
| Flavour (PF1. PF2. PF3 or PF4) | 1.00 |
| Solbrol M (Sodium salt) (Methylparaben) | 0.10 |
| 4-Hydroxy acetophenone | 0.20 |
| propylene glycol containing 5% compound of formula A | 0.80 |

The formulation as provided in Example 6, but instead of “propylene glycol containing 5% compound of formula A”, it may contain:
6 a) propanol containing 2% compound of formula A
6 b) benzyl alcohol containing 7% compound of formula A
6 c) diethylmalonate containing 3% compound of formula A
6 d) butyl lactate containing 8% compound of formula A
6 e) peppermint oil containing 1% compound of formula A

### EXAMPLE 7: TOOTHPASTE WITH ZINC CITRATE (AMOUNTS IN % B.W.)

| Ingredients | Amount |
|---|---|
| Water (deionized) | Ad 100 |
| Sorbitol 70% | 45.00 |
| Trisodiumphosphate | 0.10 |
| Saccharin | 0.20 |
| Sodiummonofluorophosphate | 1.14 |
| PEG 1500 | 5.00 |
| Sident 9 (abrasive silica) | 10.00 |
| Sident 22 S (Thickening silica) | 8.00 |
| Sodiumcarboxymethylcellulose | 1.10 |
| Zinc citrate | 1.00 |
| Titanium (IV) oxide | 0.50 |
| Water (deionized) | 4.50 |
| Sodiumlaurylsulfate (SLS) | 1.50 |
| Flavour (PF1. PF2. PF3 or PF4) | 1.00 |
| SymDiol ® 68 (1.2-Hexanediol. Caprylylglycol) | 0.25 |
| Benzyl alcohol | 0.20 |
| propylene glycol containing 5% compound of formula A | 1.00 |

The formulation as provided in Example 7, but instead of “propylene glycol containing 5% compound of formula A”, it may contain:

7 a) isopropanol containing 6% compound of formula A 7 b) dipropylene glycol containing 4% compound of formula A 7 c) glycerol containing 1% compound of formula A 7 d) 1,3-propanediol containing 5% compound of formula A 7 e) spearmint oil containing 7% compound of formula A

### EXAMPLE 8: MOUTH RINSE (AMOUNTS IN % B.W.)

| Ingredients | Amount |
|---|---|
| Ethylalcohol | 10.00 |
| Cremophor CO 40 (PEG 40 hydrogenated castor oil) | 1.00 |
| Flavour (PF1. PF2. PF3 or PF4) | 0.25 |
| Water (deionized) | Ad 100 |
| Sorbitol 70% | 5.00 |
| Sodiumsaccharin 450 | 0.07 |
| Sodiumfluoride | 0.18 |
| Benzoic acid | 0.15 |
| propylene glycol containing 5% compound of formula A | 0.25 |

The formulation as provided in Example 8, but instead of “propylene glycol containing 5% compound of formula A”, it may contain:
8 a) menthyl acetate containing 2% compound of formula A
8 b) benzyl alcohol containing 10% compound of formula A
8 c) carvacrol containing 1% compound of formula A
8 d) methylsalicylate containing 4% compound of formula A
8 e) menthone containing 5% compound of formula A

### EXAMPLE 9: GEL DENTAL CREAM (AMOUNTS IN % B.W.)

| Ingredients | Amount |
|---|---|
| Na carboxymethylcellulose | 0.40 |
| Sorbitol 70%. in water | 72.00 |
| Polyethylene glycol (PEG) 1500 | 3.00 |
| Na saccarinate | 0.07 |
| Na fluoride | 0.24 |
| Flavor (PF1. PF2. PF3 or PF4) | 1.00 |
| Abrasive silica | 11.00 |
| Thickening silica | 6.00 |
| Sodium dodecyl sulfate (SDS) | 1.40 |
| Dist. water | Ad 100 |
| 4-Hydroxyacetophenone | 0.25 |
| propylene glycol containing 5% compound of formula A | 0.80 |

The formulation as provided in Example 9, but instead of “propylene glycol containing 5% compound of formula A”, it may contain:
9 a) cinnamon oil containing 4% compound of formula A
9 b) clove oil containing 7% compound of formula A
9 c) limonene containing 2% compound of formula A
9 d) anethole containing 8% compound of formula A
9 e) carvone containing 9% compound of formula A

### EXAMPLE 10: DENTAL CREAM AGAINST PLAQUE (AMOUNTS IN % B.W.)

| Ingredients | Amount |
|---|---|
| Carrageenan | 0.90 |
| Glycerol | 15.00 |
| Sorbitol 70%. in water | 25.00 |
| PEG 1000 | 3.00 |
| Na fluoride | 0.24 |
| Tetrapotassium diphosphate | 4.50 |
| Tetrasodium diphosphate | 1.50 |
| Na saccarinate | 0.40 |
| Precipitated silica | 20.00 |
| Titanium dioxide | 1.00 |
| Triclosan | 0.30 |
| Spearmint flavor (comprising 60 wt. % I-carvone | 1.00 |
| and 25 wt. % I-menthol) | |
| Sodium dodecyl sulfate | 1.30 |
| Dist. water | Ad 100 |
| Benzylalcohol | 0.20 |
| propylene glycol containing 5% compound of formula A | 0.75 |

The formulation as provided in Example 10, but instead of “propylene glycol containing 5% compound of formula A”, it may contain:
10 a) propanol containing 3% compound of formula A
10 b) benzyl alcohol containing 7% compound of formula A
10 c) diethylmalonate containing 2% compound of formula A
10 d) spearmint oil containing 7% compound of formula A
10 e) peppermint oil containing 6% compound of formula A

### EXAMPLE 11: DENTAL CREAM FOR SENSITIVE TEETH (AMOUNTS IN % B.W.)

| Ingredients | Amount |
|---|---|
| Na carboxymethylcellulose | 0.70 |
| Xanthan gum | 0.50 |
| Glycerol | 15.00 |
| Sorbitol 70%. in water | 12.00 |
| Potassium nitrate | 5.00 |
| Sodium monofluorophosphate | 0.80 |
| Na saccharinate | 0.20 |
| Flavor (PF1. PF2. PF3 or PF4) | 1.00 |
| Ca-carbonate | 35.00 |
| Silicon dioxide | 1.00 |
| Sodium dodecyl sulfate (SDS) | 1.50 |
| Dist. water | Ad 100 |
| 4-Hydroxyacetophenone | 0.10 |
| Butyl lactate | 0.50 |
| p-Hydroxy benzaldehyde | 0.10 |
| propylene glycol containing 5% compound of formula A | 0.50 |

The formulation as provided in Example 11, but instead of “propylene glycol containing 5% compound of formula A”, it may contain:
11 a) propanol containing 5% compound of formula A
11 b) benzyl alcohol containing 8% compound of formula A
11 c) pentylene glycol containing 3% compound of formula A
11 d) ethyl lactate containing 4% compound of formula A
11 e) ethylacetate containing 1% compound of formula A

### EXAMPLE 12: TOOTH CREAM AND MOUTHWASH 2-IN-1 PRODUCT (AMOUNTS IN % B.W.)

| Ingredients | Amount |
|---|---|
| Sorbitol | 40.00 |
| Glycerol | 20.00 |
| Ethanol | 5.00 |
| Water | Ad 100 |
| Na monofluorophosphate | 0.75 |
| Saccharin | 0.20 |
| Sident 9 (abrasive silicon dioxide) | 20.00 |
| Sident 22 S (thickening silicon dioxide) | 2.00 |
| Sodium carboxymethylcellulose | 0.30 |
| Sodium lauryl sulfate (SDS) | 1.20 |
| Color (Suspension. 1% in water) C.I. Pigment Blue 15 | 0.50 |
| Flavor (PF1. PF2. PF3 or PF4) | 0.90 |
| Solbrol M, sodium salt (methylparaben, sodium salt) | 0.20 |
| propylene glycol containing 5% compound of formula A | 0.30 |

The formulation as provided in Example 12, but instead of “propylene glycol containing 5% compound of formula A”, it may contain:
12 a) dipropylene glycol containing 2% compound of formula A
12 b) 1,2-hexanediol containing 5% compound of formula A
12 c) ethanol containing 10% compound of formula A
12 d) butyl lactate containing 2% compound of formula A
12 e) peppermint oil containing 6% compound of formula A

### EXAMPLE 13: READY-TO-USE MOUTHWASH WITH FLUORIDE (AMOUNTS IN % B.W.)

| Ingredients | Amount |
|---|---|
| Ethanol | 7.00 |
| Glycerol | 12.00 |
| Na fluoride | 0.05 |
| Pluronic F-127 ® (BASF. surface-active substance) | 1.40 |
| Na phosphate buffer pH 7.0 | 1.10 |
| Na saccharinate | 0.10 |
| Flavour (PF1. PF2. PF3 or PF4) | 0.15 |
| Chlorhexidine digluconate | 0.2 |
| Dist. water | Ad 100 |
| Sorbic acid | 0.20 |
| propylene glycol containing 5% compound of formula A | 0.30 |

The formulation as provided in Example 13, but instead of “propylene glycol containing 5% compound of formula A”, it may contain:
13 a) propylene glycol containing 8% compound of formula A
13 b) ethylene glycol containing 3% compound of formula A
13 c) cinnamon oil containing 3% compound of formula A
13 d) spearmint oil containing 7% compound of formula A
13 e) peppermint oil containing 4% compound of formula A

### EXAMPLE 14: SUGAR-FREE CHEWING GUM

| Ingredients | % by weight |
|---|---|
| Chewing gum base | 30.00 |
| Sorbitol, powder | Ad 100.00 |
| Palatinite | 9.50 |
| Xylitol | 2.00 |
| Mannitol | 3.00 |
| Aspartame | 0.10 |
| Acesulfame K | 0.10 |
| Emulgum/emulsifier | 0.30 |
| Sorbitol 70%, in water | 14.00 |
| Glycerol | 1.00 |
| Flavor (PF1, PF2, PF3 or PF4) | 1.50 |
| propylene glycol containing 5% compound of formula A | 0.40 |

The formulation as provided in Example 14, but instead of “propylene glycol containing 5% compound of formula A”, it may contain:
14 a) propanol containing 6% compound of formula A
14 b) spearmint oil containing 7% compound of formula A
14 c) cinnamon oil containing 8% compound of formula A
14 d) clove oil containing 9% compound of formula A
14 e) peppermint oil containing 5% compound of formula A

### EXAMPLE 15: GELATINE CAPSULES FOR DIRECT CONSUMPTION

| Ingredients | % by weight |
|---|---|
| Gelatine shell: | |
| Glycerol | 2.014 |
| Gelatine 240 Bloom | 7.91 |
| Sucralose | 0.065 |
| Allura Red | 0.006 |
| Brilliant Blue | 0.005 |
| Core composition: | |
| Plant oil triglyceride | 74.00 |
| Aroma | 15.50 |
| propylene glycol containing 5% compound of formula A | 0.90 |

The aroma here had the following composition (data in each case in wt. %):

0.1% neotame powder, 0.05% aspartame, 29.3% peppermint oil arvensis, 29.3% peppermint piperita oil Willamette, 2.97% sucralose, 2.28% triacetin, 5.4% diethyl tartrate, 12.1% peppermint oil yakima, 0.7% ethanol, 3.36% 2-hydroxyethyl menthyl carbonate, 3.0% 2-hydroxypropyl menthyl carbonate, 0.27% vanillin, 5.5% D-limonene, 5.67% L-menthyl acetate.

The gelatine capsule, which is suitable for direct consumption, had a diameter of 5 mm, and the weight ratio of core material to shell material was 90:10. The capsules opened in the mouth within less than 10 seconds and dissolved completely within less than 50 seconds.

The formulation as provided in Example 15, but instead of “propylene glycol containing 5% compound of formula A”, it may contain:
15 a) isopropanol containing 8% compound of formula A
15 b) 1,2-hexanediol containing 5% compound of formula A
15 c) ethylbutyrate containing 3% compound of formula A
15 d) eugenol containing 2% compound of formula A
15 e) cinnamic aldehyde containing 6% compound of formula A

### EXAMPLE 16: THROAT CANDIES WITH LIQUID/VISCOUS CORE FILLING (CENTER-FILLED HARD CANDY)

| Ingredients | % by weight | % by weight |
|---|---|---|
| Mixture A (shell) (80% of the candies) | | |
| Sugar (sucrose) | Ad 100 | Ad 100 |
| Glucose syrup (solids content 80%) | 41.51 | 49.37 |
| propylene glycol containing 5% compound | 0.3 | 0.8 |
| of formula A | | |
| Aroma blend PF4 | 0.17 | 0.25 |
| I-menthol | 0.10 | — |
| Lemon oil | 0.10 | 0.10 |
| Citric acid | — | 0.91 |
| Total: | 100 | 100 |
| Mixture B (core) (20% of the candies) | | |
| High fructose maize syrup (sugar solids | 84.38 | 84.36 |
| content 85%, only 15% water) | | |
| Glycerol | 15.0 | 15.0 |
| Lecithin | 0.02 | 0.02 |
| Cinnamon oil | — | 0.32 |
| Spearmint oil | 0.28 | — |
| Capsaicin | 0.05 | — |
| Vanillyl alcohol n-butyl ether | — | 0.10 |
| Red dye, as 5% aqueous solution | 0.20 | 0.20 |
| Vanillin | 0.07 | — |
| Total | 100 | 100 |

The formulation as provided in Example 16, but instead of “propylene glycol containing 5% compound of formula A”, it may contain:
16 a) benzyl alcohol containing 10% compound of formula A
16 b) diethylmalonate containing 7% compound of formula A
16 c) butyl lactate containing 3% compound of formula A
16 d) spearmint oil containing 7% compound of formula A
16 e) peppermint oil containing 4% compound of formula A

Candies with a liquid/viscous core were produced on the basis of the methods described in U.S. Pat. No. 6,432,441 and those described in U.S. Pat. Nos. 5,458,894 or 5,002,791. The two mixtures A and B were separately processed to form bases for the shell (mixture A) or core (mix-ture B). When consumed by affected individuals, the filled throat candies obtained by means of coextrusion were effective against coughing, sore throat and hoarseness.

### EXAMPLE 17: COMPRESSED TABLETS FOR CONSUMPTION

| Ingredients | % by weight |
|---|---|
| Magnesium stearate (as lubricant) | 0.90 |
| Citric acid | 0.20 |
| propylene glycol containing 5% compound of formula A | 0.50 |
| Dextrose | Ad 100 |

The formulation as provided in Example 17, but instead of “propylene glycol containing 5% compound of formula A”, it may contain:
17 a) propanol containing 3% compound of formula A
17 b) anethole containing 7% compound of formula A
17 c) methylsalicylate containing 5% compound of formula A
17 d) carvone containing 6% compound of formula A
17 e) peppermint oil containing 1% compound of formula A

1. A method for altering the bacterial composition of an oral biofilm such that the proportion of microorganisms detrimental to oral health is reduced while the proportion of health-promoting microorganisms is increased, the method comprising:
treating an oral biofilm with an oral composition comprising:
i) a compound in accordance with formula 1 or salt thereof 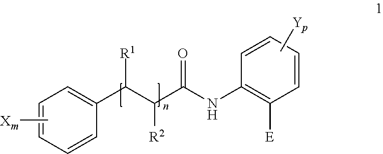
wherein the compound in accordance with formula 1 satisfies the following parameters:
m=0, 1, 2, or 3,
p=0, 1 or 2,
n=0, 1, or 2, and
wherein if n=1 or 2 then R¹ and R² each denote H or together form a further chemical bond,
if m=1, 2, or 3 then each X, independently of the other, denotes OH, Oalkyl, or Oacyl,
if p=1 or 2 then each Y, independently of the other, denotes OH, Oalkyl or Oacyl,
E=H or denotes a radical —COOR³,
R³═H or alkyl, in which R³═H also for the corresponding salts, and
ii) a suitable carrier.

2. The method according to claim 1, wherein the compound in accordance with formula 1 satisfies the following parameters: n=0, E=—COOH and m+p≤3.

3. The method according to claim 2, wherein the compound in accordance with formula 1 satisfies the following parameters m+p=0.

4. The method according to claim 1, wherein the proportions of one or more of the bacteria selected from Prevotella, Veillonella, Porphyromonas, Atopobium, Selenomonas and Fusobacterium is reduced and the proportions of one or more of the bacteria selected from Neisseria, Rothia, Corynebacterium and Streptococcus is increased, in each case with respect to a negative control.

5. The method according to claim 1, wherein the carrier is selected from oils, alcohols, diols, polyols, phenols, and esters.

6. The method according to claim 1, wherein a total amount of the compound in accordance with formula 1 is in the range from 0.0005 to 1 wt. %, with respect to the total weight of the oral composition.

7. The method according to claim 1, wherein a total amount of the compound in accordance with formula 1 is sufficient to reduce the proportions of one or more of the bacteria selected from Prevotella, Veillonella, Porphyromonas, Atopobium, Selenomonas and Fusobacterium and to increase the proportions of one or more of the bacteria selected from Neisseria, Rothia, Corynebacterium and Streptococcus, in each case with respect to a negative control.

8. The method according to claim 1, wherein the oral composition is coated or encapsulated.

9. The method according to claim 7, wherein the oral biofilm is treated with an oral care product comprising the oral composition, the oral care product selected from tooth paste, tooth powder, tooth gel, tooth cleaning liquid, tooth cleaning foam, mouth wash, mouth rinse, mouth spray, dental floss, chewing gum and lozenges.

10. An oral composition comprising:
i) a compound in accordance with formula A, 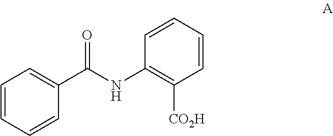 [SMILES: O=C(Nc1ccccc1C(=O)O)c1ccccc1]
and
ii) a carrier selected from oils, alcohols, diols, polyols, phenols, and esters.

11. The method of claim 4, wherein the method is effective to treat halitosis.

12. The method of claim 11, wherein Solobacterium moorei is suppressed in the oral biofilm.

13. The oral composition of claim 10, wherein the oral composition is effective to reduce proportions of one or more of the bacteria selected from Prevotella, Veillonella, Porphyromonas, Atopobium, Selenomonas and Fusobacterium and increase proportions of one or more of the bacteria selected from Neisseria, Rothia, Corynebacterium and Streptococcus, in each case with respect to a negative control.

14. The oral composition of claim 10, wherein the oral composition is coated or encapsulated.

15. The oral composition of claim 10, wherein an amount of the compound in accordance with formula A ranges from 1 to 25 wt. % with respect to an amount of the carrier.

16. An oral care product being selected from tooth paste, tooth powder, tooth gel, tooth cleaning liquid, tooth cleaning foam, mouth wash, mouth rinse, mouth spray, dental floss, chewing gum and lozenges, the oral care product comprising the oral composition of claim 10.

17. The oral care product of claim 16, further comprises one or more compounds selected from sodium fluoride, sodium monofluoro phosphate, tin difluoride, quarternary ammonium fluorides, zinc citrate, zinc sulfate, tin pyrophosphate, tin dichloride, pyrophosphates, triclosane, cetyl pyridinium chloride, aluminum lactate, potassium citrate, potassium nitrate, potassium chloride, strontium chloride, hydrogen peroxide, aroma substances, and sodium bicarbonate.

18. The oral care product of claim 16, further comprises one or more compounds selected from saccharine, inositol phosphate, nucleotides, guanosine monophosphate, adenosine monophosphate or other similar compounds, sodium glutamate, and phenoxy propionic acid.

19. The oral care product of claim 16, further comprises one or more compounds selected from polyvinyl acetate (PVA), polyethylene, low or medium molecular weight polyiso butane (PIB), polybutadiene, isobutene/isoprene copolymers, polyvinyl ethyl ether (PVE), polyvinyl butyl ether, copolymers of vinyl esters and vinyl ethers, styrene/butadiene copolymers (SBR), vinyl elastomers, and mixtures thereof.
